(19) United States
(12) Patent Application Publication
(10) Pub. No.: US 2017/0328540 A1
(43) Pub. Date: Nov. 16, 2017

(54) LIGHTING DEVICE HAVING A WAVELENGTH CONVERSION ASSEMBLY

(71) Applicant: OSRAM GmbH, Munich (DE)

(72) Inventors: Ruediger Paul, Ottobrunn (DE); Stefan Hadrath, Falkensee (DE)

(21) Appl. No.: 15/521,916

(22) PCT Filed: Oct. 5, 2015
(86) PCT No.: PCT/EP2015/072921

§ 371 Date: Apr. 26, 2017

(30) Foreign Application Priority Data
Oct. 29, 2014 (DE) 10 2014 222 130.7

(51) Int. Cl. F21V 9/16; H04N 9/31; G02B 26/00; A61B 1/06

(52) U.S. Cl. CPC F21V 9/16; H04N 9/3158; A61B 1/0661; G02B 26/008; H04N 9/3117

(57) ABSTRACT

A lighting device is disclosed with excitation light source(s) for emitting excitation light along an excitation light path; a wavelength conversion assembly including wavelength conversion element(s) for converting the excitation light into conversion light and emitting it into the same half-space from which the excitation light is radiated onto the surface of the element, and reflection element(s) for reflecting, in unconverted fashion, the excitation light intermittently radiated onto the reflection element from the source(s) along the portion of the excitation light path onto a reflection light path as reflection light; and a dichroic mirror for deflecting the excitation light coming from the source(s) onto the portion of the excitation light path on which the excitation light is radiated onto the wavelength conversion element(s) or the reflection element(s). The mirror is configured such that the conversion light is transmitted through the mirror and the reflection light is guided past the mirror.

### RELATED APPLICATIONS

The present application is a national stage entry according to 35 U.S.C. §371 of PCT application No.: PCT/EP2015/072921 filed on Oct. 5, 2015, which claims priority from German application No.: 10 2014 222 130.7 filed on Oct. 29, 2014, and is incorporated herein by reference in its entirety.

### TECHNICAL FIELD

The present disclosure relates to a lighting device including an excitation light source for emitting primary radiation which is utilizable as excitation light and a wavelength conversion assembly for converting the excitation light into light in a spectral range which differs from the excitation light (conversion light).

### BACKGROUND

Light sources with a high luminance may be used, for example, in the field of endoscopy or in projection appliances, with gas discharge lamps currently still being used most widely to this end. More recent developments are focused on combining an excitation light source with a high power density, e.g. a laser, with a phosphor element arranged at a distance therefrom. The present disclosure is also applicable to lighting devices in the entertainment sector, for example for stage lighting and/or image projection.

The prior art has disclosed such lighting devices which include a wavelength conversion element in the form of a phosphor element. Here, these lighting devices include an excitation light source which excites the phosphor to emit light at a wavelength which differs from the excitation light wavelength. In particular, use is also made of excitation light in the blue spectral range. By way of a suitable deflection of the blue excitation light and the conversion light emitted by the phosphor, it is possible to combine these two light paths and feed these to an optical integrator.

In particular, a phosphor wheel may also be provided as a wavelength conversion assembly, said phosphor wheel rotating about an axis of rotation and, in the process, being irradiated by excitation light on a circular track. Here, different colored phosphors may also be successively arranged in the circumferential direction on the phosphor wheel such that a temporal sequence of conversion light with different colors, e.g. red (R), green (G) and blue (B) light, is produced. Then, colors of the conversion light together sequentially span an RGB color space.

Document CN 102385233 A discloses a lighting device for a projector, including an excitation laser, a phosphor wheel for converting the wavelength of the excitation laser light into conversion light and a filter wheel for spectral filtering of the conversion light. The filter wheel and the phosphor wheel are arranged on a common shaft and thus rotate at the same speed. The excitation laser light is reflected onto the phosphor wheel with the aid of a dichroic mirror. By contrast, the conversion light radiated back by the phosphor wheel passes through the dichroic mirror and is subsequently incident on the filter wheel. A transparent segment in the phosphor wheel allows the excitation laser light to pass the phosphor wheel in a spectrally unchanged fashion and the latter is guided to the dichroic mirror by way of a so-called wrap-around loop and brought together with the conversion light path. The wrap-around loop necessitates further optical elements which, moreover, increase the external dimensions of the lighting device.

### SUMMARY

The object of the present disclosure is to specify an alternative lighting device for using the excitation light and the conversion light which, moreover, makes do with as few components as possible.

A further aspect of the present disclosure lies in a design of the lighting device which is as compact as possible.

This object is achieved by a lighting device for producing light by means of a wavelength conversion assembly, including at least one excitation light source configured to emit excitation light along an excitation light path, a wavelength conversion assembly which is arranged in the excitation light path and includes at least one wavelength conversion element configured to at least partly convert into conversion light the excitation light at least intermittently radiated onto the wavelength conversion element from the at least one excitation light source along a portion of the excitation light path and emit the conversion light into the same half-space from which the excitation light is radiated onto the surface of the wavelength conversion element, and at least one reflection element configured to reflect, at least partly in unconverted fashion, the excitation light at least intermittently radiated onto the reflection element from the at least one excitation light source along the portion of the excitation light path onto a reflection light path as reflection light, a dichroic mirror for deflecting the excitation light coming from the at least one excitation light source onto the portion of the excitation light path which the excitation light is radiated onto the at least one wavelength conversion element or the at least one reflection element, wherein the dichroic mirror is arranged and configured in such a way that the conversion light is transmitted through the dichroic mirror and the reflection light on the reflection light path is guided past the dichroic mirror.

Particularly advantageous configurations are found in the dependent claims.

The basic idea of the present disclosure consists of guiding both the conversion light converted by a conversion element and the excitation light reflected in unconverted fashion by a reflection element on a common light path. To this end, the excitation light coming from a first direction is mirrored sequentially in time onto the conversion element or the reflection element along a second direction by way of a dichroic mirror. The dichroic mirror is configured to transmit the conversion light coming from the conversion element. The reflection light coming from the reflection element is guided past the dichroic mirror. There is no provision for separation into a separate conversion light path and a path for the unconverted excitation light (reflection light in this case), as is disclosed in the prior art. As a result, it is possible to dispense with the optical components required for a separate path for the unconverted excitation light, for example a wrap-around loop.

Advantageously, blue light (i.e. light in the blue spectral range), in particular blue laser light, is used as excitation light as the excitation light then may be used additionally in unconverted fashion as a blue color channel (reflection light) as well, in addition to exciting a wavelength conversion element, for example phosphor.

Advantageously, a collecting optical unit is optically arranged between the dichroic mirror and the wavelength conversion assembly. The collecting optical unit is configured firstly to focus the excitation light of the excitation light source onto the wavelength conversion assembly and secondly to collect and collimate the conversion light emitted by the wavelength conversion element of the wavelength conversion assembly and the reflection light reflected in unconverted fashion by the reflection element. In the simplest case, the collecting optical unit may be embodied as a converging lens, but it may also be embodied as a lens system or any other optical element with the aforementioned optical effect.

Moreover, the dichroic mirror is advantageously arranged in such a way that the excitation light incident on the dichroic mirror from the excitation light source is reflected (excitation light path) onto the collecting optical unit in a manner offset to the optical axis (off-axis) thereof. Finally, the excitation light source, the dichroic mirror, the collecting optical unit and the reflection element are configured and arranged in such a way that the reflection light path extends parallel to the excitation light path between the dichroic mirror and the collecting optical unit, i.e. the reflection light is likewise mirrored back off-axis—but past the dichroic mirror.

As a result, the reflection light and the conversion light use the same light path and may be focused in an optical integrator for application-dependent further use, for example by way of a further collecting optical unit. The optical integrator homogenizes the incident light beams, for example by multiple reflection on the path from the integrator input to the integrator output.

Optionally, a color filter or color filter wheel may be arranged between the further (second) collecting optical unit and the optical integrator in order to improve the color purity of the respective colored conversion light (e.g. red, green, yellow, etc.). To this end, the color filter wheel may include color filter segments which correspond to, and are synchronized with, the phosphor segments of the phosphor wheel. During the reflection phase, provision may be made of a segment which leaves the excitation light spectrally unmodified, which rotates through the focus of the second collecting optical unit.

In place of the second collecting optical unit, provision may also be made, where necessary, of a different optical element or further optical elements, for example a mirror element for deflecting the common light path in order to adapt the geometric form of the lighting device, or the like.

The wavelength conversion assembly is configured for the excitation light to be radiable onto the at least one reflection element or the at least one wavelength conversion element in a temporally sequential sequence.

Advantageously, the wavelength conversion assembly is embodied as a body which is rotatable about an axis, the at least one wavelength conversion element and the at least one reflection element being arranged on said body in such a way that the at least one wavelength conversion element and the at least one reflection element are moved through the excitation light path in succession when the body is rotated. In this way, it is possible to provide a temporal sequence of conversion light (excitation light incident on conversion element) and non-converted reflection light (excitation light incident on reflection element).

By way of example, the wavelength conversion assembly may be embodied as a roller which is rotatable about an axis of rotation, with the at least one wavelength conversion element and the at least one reflection element being arranged on the lateral surface thereof, in particular in a sequential sequence.

Advantageously, the wavelength conversion assembly is embodied as a phosphor wheel which is rotatable about an axis of rotation of the phosphor wheel. The at least one wavelength conversion element may be arranged in at least one segment of a ring-shaped region of the phosphor wheel extending around the axis of rotation of the phosphor wheel. Equally, the at least one reflection element may be arranged in at least one segment of a ring-shaped region of the phosphor wheel extending around the axis of rotation of the phosphor wheel. The at least one reflection element may be embodied as an area, for example as a mirror area, at least partly reflecting the excitation light.

A phosphor layer, for example a yellow phosphor which converts blue excitation light into yellow light, may be provided for the wavelength conversion element. Light which, in the temporal mean, appears white to the human eye may be produced in the case of a superposition and mixture of the temporally sequential sequence of both colored light components, it being possible to set the color temperature of said light, for example by the targeted selection of the respective temporal components of blue and yellow light or by setting an intensity of the incident excitation light, in particular during the reflection phases for controlling the blue light component. By way of example, the wavelength conversion assembly may include a red phosphor segment and a green phosphor segment for a sequential colored light production. A sequence of red, green and blue light may be produced therewith with the aid of a reflection element and blue light as excitation light. It is also possible to use other phosphors or further phosphors, for example a yellow phosphor, phosphors with different color nuances, for example two different red phosphors or green phosphors, etc., where necessary.

The wavelength conversion assembly may also be embodied as a body which is displaceable to and fro along an axis, with the at least one wavelength conversion element and the at least one reflection element being arranged on said body in such a way that the at least one wavelength conversion element and the at least one reflection element are successively moved through the excitation light path when the body is displaced.

Advantageously, the excitation light source includes at least one laser diode. In order to be able to provide the high excitation light power required for many applications, it may be advantageous to attach a plurality of laser diode chips in a common housing. Each laser diode may be equipped with at least one dedicated and/or common optical unit (“multi-lens array”) for beam guidance, e.g. equipped with at least one Fresnel lens, collimator, etc. Other excitation light sources are also conceivable, such as e.g. those which include superluminescent diodes, LEDs, organic LEDs or the like.

The use of the lighting device according to the present disclosure, as described above, is also claimed for at least one of the following applications: video projection, endoscopy, light projection for entertainment purposes, room lighting, industrial and medical applications.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the disclosed embodiments. In the following description, various embodiments described with reference to the following drawings, in which:

FIG. 1 shows an embodiment of a lighting device according to the present disclosure, including a phosphor wheel in a reflection light phase,

FIGS. 2A, 2B show a top view and a sectional view, respectively, of the phosphor wheel from FIG. 1 in a position corresponding to the reflection light phase,

FIG. 3 shows the embodiment from FIG. 1 in a conversion light phase,

FIGS. 4A, 4B show a top view and a sectional view, respectively, of the phosphor wheel from FIG. 3 in a position corresponding to the conversion light phase,

FIG. 5 shows an embodiment of an excitation light source for a lighting device according to the present disclosure.

### DETAILED DESCRIPTION

The same or equivalent features may also be denoted by the same reference sign below for reasons of simplicity.

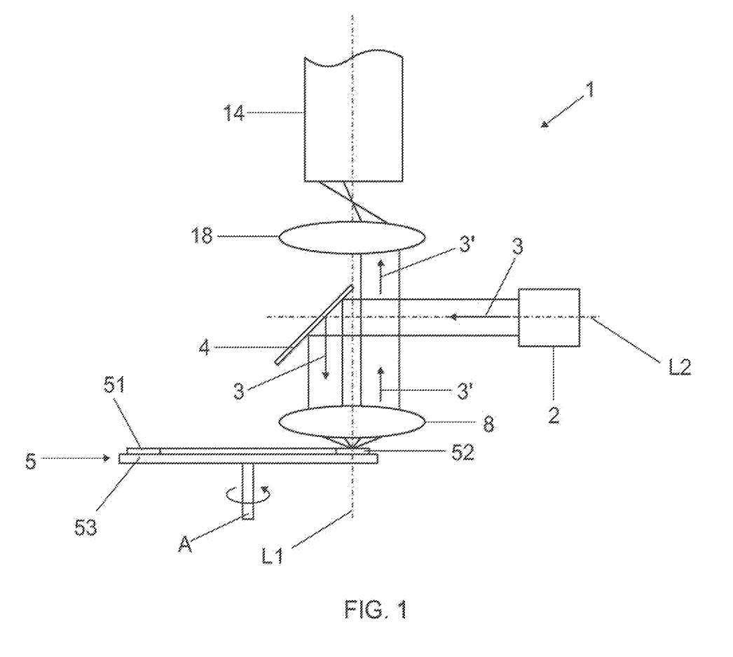

FIG. 1 shows a schematic illustration of a lighting device 1 in accordance with one embodiment of the present disclosure. The lighting device 1 includes an excitation light source 2 embodied as a laser device. The excitation light 3 is also concomitantly used as blue color channel. Hence, the excitation light source 2 is configured to emit excitation light 3 in the blue spectral range, for example in the range of 440-470 nm, particularly advantageously at approximately 450 nm. Moreover, this is a suitable excitation wavelength for many phosphors.

The blue laser light 3 of the excitation light source 2, which is advantageously at least approximately collimated in the direction of an optical axis L2, is deflected by means of a dichroic mirror 4 onto a wavelength conversion assembly embodied as a phosphor wheel 5. To this end, the dichroic mirror 4 has a coating which mirrors the laser light 3 but is transparent to the longer wave spectrum of the visible light.

Moreover, the blue laser light 3 is focused onto the surface of the phosphor wheel 5 facing the incident excitation light 3 with the aid of a first collecting optical unit 8 arranged between dichroic mirror 4 and phosphor wheel 5. Here, excitation light source 2, dichroic mirror 4 and first collecting optical unit 8 are adjusted in relation to one another in such a way that the blue laser light 3 (symbolized by an arrow) is incident on the first collecting optical unit 8 with a parallel offset from the optical axis L1 thereof (off-axis beam path).

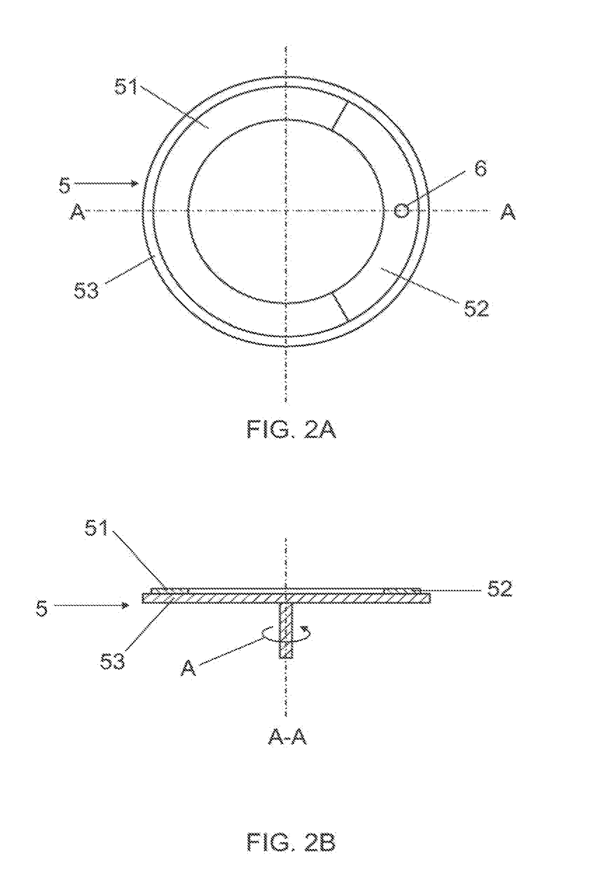

Below, reference is now also made to FIG. 2A, which shows the phosphor wheel 5 in the orientation in accordance with FIG. 1 in a plan view, and FIG. 2B, which shows a schematic cross section along the line AA. The phosphor wheel 5 includes a circular-disk-shaped carrier 53 which is mounted in rotatable fashion about the axis of rotation A. The side of the carrier 53 facing the incident excitation light 3 is provided with a circular-ring-segment-shaped wavelength conversion element 51 which is embodied as a yellow phosphor layer. Moreover, the carrier 53 includes a reflection element 52 embodied as a circular-ring-segment-shaped mirror area which adjoins the wavelength conversion element 51 and reflects blue light in a spectrally unmodified manner. By way of example, the mirror area 52 may be embodied by a segment of the advantageously mirrored surface of the carrier 53 which has not been coated by phosphor. The laser spot radiated onto the mirror area 52 by the incident excitation light is symbolized as a small circular area 6.

The lighting device 1 depicted in FIG. 1 is thus provided for a temporally sequential sequence of yellow conversion light (Y) and blue reflection light (B). By way of example, it is suitable as a temporally averaged white light source for the human eye. Moreover, further or other phosphor segments may also be provided where necessary, for example, additionally or alternatively, phosphor segments with a green phosphor layer (for green conversion light G) and/or red phosphor layer (for red conversion light R) for an RGB or RGBY light source. Likewise, provision may also be made of more than one reflection element.

FIG. 1 depicts the temporal phase during which the mirror segment 52 of the phosphor wheel 5 rotates through the focus of the blue laser light 3 (reflection light phase). During the reflection light phase, the incident blue laser light 3 is reflected back without conversion by the mirror segment 52 of the phosphor wheel 5. The reflected laser light 3′ (reflection light; likewise symbolized by an arrow) is guided back in a collimated fashion which is mirror imaged to the incident blue laser light 3, i.e. parallel thereto, by the first collecting optical unit 8 (off-axis beam path). So that the blue reflection light beam 3′ may be guided past the blue-light reflecting dichroic mirror 4 without impediment, the dichroic mirror 4 has a sufficiently short embodiment or is arranged in such a way that it does not block the reflection light path. Hence, the collimated reflection light 3′ reaches past the dichroic mirror 4 onto a second collecting optical unit 18. The second collecting optical unit 18 guides the reflection light 3′ into an optical integrator 14.

By way of example, the optical integrator 14 is a suitable glass rod which spatially homogenizes the sequential blue and yellow light on the basis of multiple total-internal reflections and—when considered integrated over time—mixes said light to form white mixed light for the human eye.

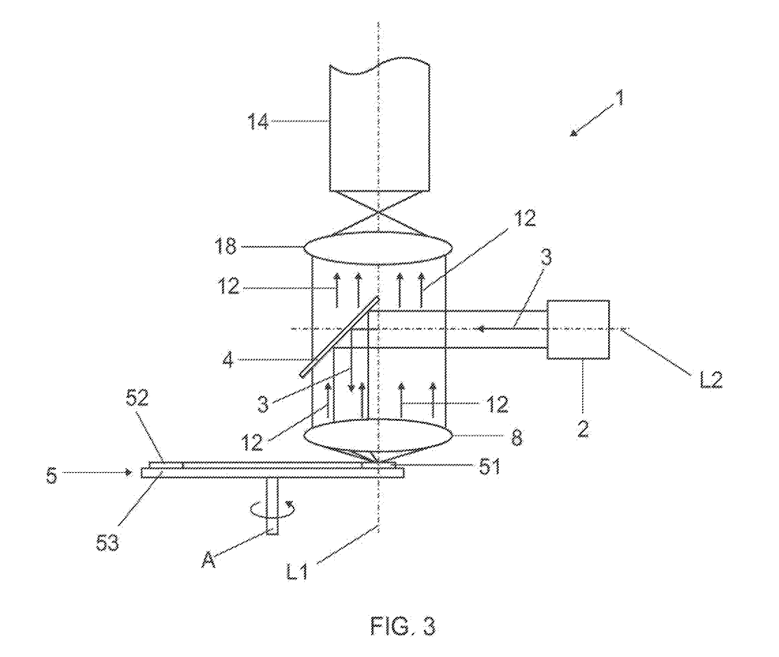

FIG. 3 depicts a conversion light phase of the lighting device 1, during which the yellow phosphor segment 51 of the phosphor wheel 5 rotates through the (excitation) light path of the blue laser light 3.

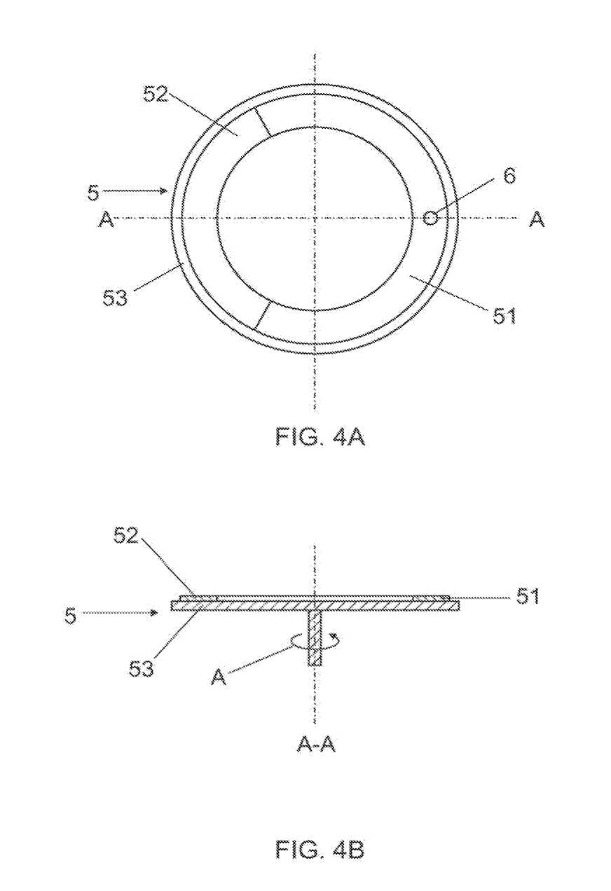

Below, reference is also made to FIGS. 4A, 4B, which show the phosphor wheel 5 already shown in FIG. 2 in the orientation in accordance with FIG. 3 in this case, namely rotated on through 180°. FIG. 4A once again shows a plan view; FIG. 4B shows a schematic cross section along the line AA.

The blue laser light 3 is converted into conversion light in the yellow spectral range (also referred to, in short, as “yellow conversion light” (12) below) by the yellow phosphor of the wavelength conversion element 51 during the conversion light phase. To this end, the blue laser light 3 deflected by the dichroic mirror 4 is focused onto the wavelength conversion element 51 by means of the first collecting optical unit 8 and said blue laser light produces the laser spot 6 there (see FIG. 4). The blue laser light incident within the laser spot 6 is converted into yellow conversion light 12 by the yellow phosphor and emitted into the same half-space from which the excitation light 3 radiates onto the surface of the wavelength conversion element 51, approximately with a Lambert distribution. The conversion light 12 is collected and collimated by the first collecting optical unit 8. Since the wavelength conversion element 51 in this case rotates perpendicularly through the local optical axis L1 of the excitation light path, the principal direction of the Lambert distribution coincides with the surface normal of the wavelength conversion element 51 and the local optical axis L1 of the excitation light path. Therefore, the collimated conversion light 12 extends parallel to the incoming excitation light 3 in the opposite direction, is transmitted to the dichroic mirror 4 and is thereupon guided into the optical integrator 14 by way of the second collecting optical unit 18.

The light emitted by the optical integrator 14 is perceived by the human eye as mixed light with yellow (conversion light 12) and blue (reflection light 3′) colored light components in the case of light sequences that are carried out sufficiently quickly, e.g. in the case of a rotation of the phosphor wheel 5 of at least 25 revolutions per second.

As a result of the lateral coupling-in of the excitation light 3 via the dichroic mirror 4 which is arranged off axis and which reflects blue light, it is possible to guide both the reflection light 3′ and the conversion light 12 over the same light path. As a result, the same optical elements 8, 18 may be used for the reflection light 3′ and the conversion light 12.

Consequently, the optical structure is very compact and makes do with relatively few optical elements 4, 8, 18.

By way of example, in order to improve the color purity of the respective colored conversion light (e.g. red, green, yellow, etc.), in particular for projection applications, it is possible to arrange a filter wheel (not depicted here) between the second collecting optical unit 18 and the optical integrator 14. To this end, color filter segments corresponding to, and synchronized with, the phosphor segments of the phosphor wheel 5 should be provided. During the reflection light phase, a segment leaving the blue light spectrally unchanged rotates through the focus of the second collecting optical unit 13. This blue light segment may also be embodied as a color-neutral optical scattering element in order to reduce coherence effects (speckle).

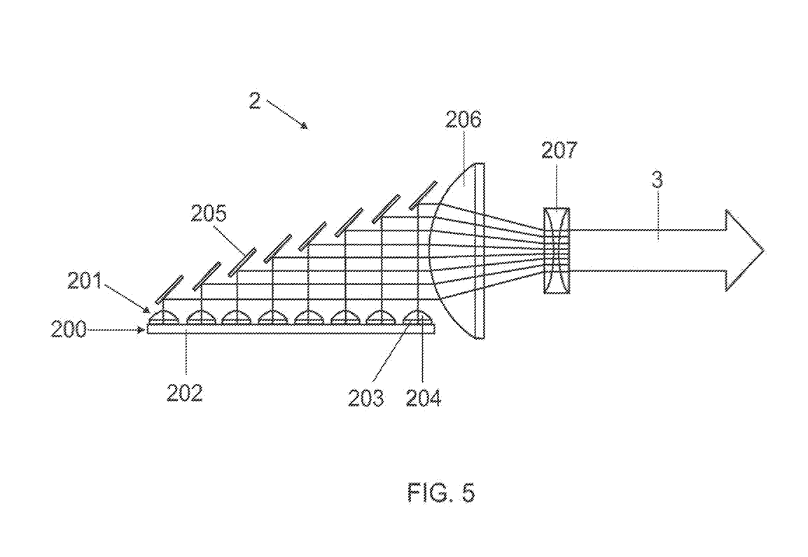

FIG. 5 shows a schematic illustration of a possible embodiment of the excitation light source 2 only indicated symbolically in the above exemplary embodiment of the present disclosure. Here, the excitation light source 2 includes a light source 200 which is embodied as a laser diode matrix and which includes a multiplicity of laser diodes 201. The arrangement of the laser diodes 201 does not only extend along one row, as may be identified in FIG. 5, but also into the plane of the drawing in a matrix-like manner. To this end, the individual laser diodes 201 are arranged on a common carrier plate 202. Each laser diode 201 is provided with a primary lens 204. The primary lenses 204 in each case serve to collimate the laser radiation emitted by the associated chip 203. Alternatively, a single-part lens matrix (“multi-lens array”) may also be provided instead of the individual primary lenses 204, a corresponding collimation lens being integrated for each chip in said single-part lens matrix (not depicted here). The collimated laser rays of the individual laser diodes 201 are deflected with the aid of elongate mirror elements 205, arranged in a step-like manner, into a common direction perpendicular to the emission direction of the laser diodes 201. As a result, the spatial extent of the laser beam is compressed along the axis of the laser diode matrix 200 lying in the plane of the drawing. A further compression of the laser beam is carried out by the collecting lens 206 disposed downstream thereof. The concave lens system 207 following thereafter produces a collimated laser beam 3 which is symbolized by the wide arrow. Thus, the lenses 206 and 207 form a telescope.

The present disclosure proposes a lighting device (1) including an excitation light source (2) and a wavelength conversion assembly (5), wherein the wavelength conversion assembly (5) includes a conversion element (51) and a reflection element (52) and is configured in such a way that the excitation light (3) is not only wavelength-converted into conversion light but, at a different time, additionally reflected in an unconverted fashion as reflection light (3′) into the same light path as the conversion light. To this end, the excitation light (3) coming from the side is mirrored temporally in succession onto the conversion element (51) and the reflection element (52), respectively, of the wavelength conversion assembly (5) by way of a dichroic mirror (4). The dichroic mirror (4) is configured to be transmissive for the conversion light coming from the conversion element (51). The reflection light (3′) coming from the reflection element (52) is guided past the dichroic mirror (4). Reflection light (3′) and conversion light may be forwarded by way of a common optical unit (18) disposed downstream of the dichroic mirror (4) into an optical integrator (14).

While the disclosed embodiments have been particularly shown and described with reference to specific embodiments, it should be understood by those skilled in the art that various changes in form and detail may be made therein without departing from the spirit and scope of the disclosed embodiments as defined by the appended claims. The scope of the disclosed embodiments is thus indicated by the appended claims and all changes which come within the meaning and range of equivalency of the claims are therefore intended to be embraced.

1. A lighting device for producing light by means of a wavelength conversion assembly, comprising
at least one excitation light source configured to emit excitation light along an excitation light path,
a wavelength conversion assembly which is arranged in the excitation light path and comprises
at least one wavelength conversion element configured to at least partly convert into conversion light the excitation light at least intermittently radiated onto the wavelength conversion element from the at least one excitation light source along a portion of the excitation light path and emit the conversion light into the same half-space from which the excitation light is radiated onto the surface of the wavelength conversion element, and
at least one reflection element configured to reflect, at least partly in unconverted fashion, the excitation light at least intermittently radiated onto the reflection element from the at least one excitation light source along the portion of the excitation light path onto a reflection light path as reflection light, and
a dichroic mirror for deflecting the excitation light coming from the at least one excitation light source onto the portion of the excitation light path on which the excitation light is radiated onto the at least one wavelength conversion element or the at least one reflection element,
wherein the dichroic mirror is arranged and configured in such a way that the conversion light is transmitted through the dichroic mirror and the reflection light on the reflection light path is guided past the dichroic mirror.

2. The lighting device as claimed in claim 1, further comprising a collecting optical unit optically arranged between the dichroic mirror and the wavelength conversion assembly and configured firstly to focus the excitation light of the excitation light source onto the wavelength conversion assembly and secondly to collect and collimate the conversion light emitted by the wavelength conversion element and the reflection light reflected by the reflection element.

3. The lighting device as claimed in claim 2, wherein the dichroic mirror is arranged in such a way that the excitation light is reflected onto the collecting optical unit in a manner offset to the optical axis thereof.

4. The lighting device as claimed in claim 3, wherein the excitation light source, the dichroic mirror, the collecting optical unit and the reflection element are configured and arranged in such a way that the excitation light path extends parallel to the reflection light path between the dichroic mirror and the collecting optical unit.

5. The lighting device as claimed in claim 1, wherein the wavelength conversion assembly is embodied as a body which is rotatable about an axis, the at least one wavelength conversion element and the at least one reflection element being arranged on the body in such a way that the at least one wavelength conversion element and the at least one reflection element move through the excitation light path in succession when the body is rotated.

6. The lighting device as claimed in claim 5, wherein the wavelength conversion assembly is embodied as a phosphor wheel which is rotatable about an axis of rotation of the phosphor wheel, wherein the at least one wavelength conversion element is arranged in at least one segment of a ring-shaped region of the phosphor wheel extending around the axis of rotation of the phosphor wheel.

7. The lighting device as claimed in claim 6, wherein the at least one reflection element is arranged in at least one segment of a ring-shaped region of the phosphor wheel extending around the axis of rotation of the phosphor wheel.

8. The lighting device as claimed in claim 1, further comprising a second collecting optical unit optically arranged downstream of the dichroic mirror and configured to collect the conversion light and the reflection light.

9. The lighting device as claimed in claim 8, further comprising an optical integrator optically arranged downstream of the second collecting optical unit for feeding the conversion light and the reflection light.

10. A use of a lighting device comprising:
emitting excitation light along an excitation light path by at least one excitation light source;
arranging a wavelength conversion assembly in the excitation light path, wherein the wavelength conversion assembly comprises,
at least one wavelength conversion element configured to at least partly convert into conversion light the excitation light at least intermittently radiated onto the wavelength conversion element from the at least one excitation light source along a portion of the excitation light path and emit the conversion light into the same half-space from which the excitation light is radiated onto the surface of the wavelength conversion element, and
at least one reflection element configured to reflect, at least partly in unconverted fashion, the excitation light at least intermittently radiated onto the reflection element from the at least one excitation light source along the portion of the excitation light path onto a reflection light path as reflection light; and
deflecting, by a dichroic mirror, the excitation light coming from the at least one excitation light source onto the portion of the excitation light path on which the excitation light is radiated onto the at least one wavelength conversion element or the at least one reflection element, wherein the dichroic mirror is arranged and configured in such a way that the conversion light is transmitted through the dichroic mirror and the reflection light on the reflection light path is guided past the dichroic mirror.

11. The use of a lighting device as claimed in claim 10, further comprising optically arranging a collecting optical unit between the dichroic mirror and the wavelength conversion assembly, wherein the collecting optical unit is configured firstly to focus the excitation light of the excitation light source onto the wavelength conversion assembly and secondly to collect and collimate the conversion light emitted by the wavelength conversion element and the reflection light reflected by the reflection element.

12. The use of a lighting device as claimed in claim 11, wherein the dichroic mirror is arranged in such a way that the excitation light is reflected onto the collecting optical unit in a manner offset to the optical axis thereof.

13. The use of a lighting device as claimed in claim 12, wherein the excitation light source, the dichroic mirror, the collecting optical unit and the reflection element are configured and arranged in such a way that the excitation light path extends parallel to the reflection light path between the dichroic mirror and the collecting optical unit.

14. The use of a lighting device as claimed in claim 10, wherein the wavelength conversion assembly is embodied as a body which is rotatable about an axis, the at least one wavelength conversion element and the at least one reflection element being arranged on the body in such a way that the at least one wavelength conversion element and the at least one reflection element move through the excitation light path in succession when the body is rotated.

15. The use of a lighting device as claimed in claim 14, wherein the wavelength conversion assembly is embodied as a phosphor wheel which is rotatable about an axis of rotation of the phosphor wheel, wherein the at least one wavelength conversion element is arranged in at least one segment of a ring-shaped region of the phosphor wheel extending around the axis of rotation of the phosphor wheel.

16. The use of a lighting device as claimed in claim 15, wherein the at least one reflection element is arranged in at least one segment of a ring-shaped region of the phosphor wheel extending around the axis of rotation of the phosphor wheel.

17. The use of a lighting device as claimed in claim 10, further comprising optically arranging a second collecting optical unit downstream of the dichroic mirror, wherein the second collecting optical unit is configured to collect the conversion light and the reflection light.

18. The use of a lighting device as claimed in claim 17, further comprising optically arranging an optical integrator downstream of the second collecting optical unit for feeding the conversion light and the reflection light.
